(19) United States
(12) Patent Application Publication
(10) Pub. No.: US 2010/0233258 A1
(43) Pub. Date: Sep. 16, 2010

(54) TABLET HAVING CONCAVITY WITH ACTIVE INGREDIENT DISPOSED THEREIN AND MANUFACTURING METHOD THEREOF

(75) Inventor: Chaur-Ming Jan, Coral Springs, FL (US)

Correspondence Address: Byrne Poh LLP, 11 Broadway, Ste 865, New York, NY, 10004, US

(73) Assignee: Inovista Inc., Coral Springs, FL (US)

(21) Appl. No.: 12/404,271

(22) Filed: Mar. 13, 2009

(51) Int. Cl. A61K 9/20; A61K 31/565; A61K 31/57; A61K 31/495; A61K 31/192; A61K 31/485

(52) U.S. Cl. 424/465; 514/182; 514/170; 514/178; 514/255.04; 514/570; 514/282

(57) ABSTRACT

The present invention provides a method of manufacturing tablets. An example of the method involves preparing a placebo blend containing only excipients. This placebo blend is compressed into tablets having concavities such as pinholes or indentations. Separately, a dispersion containing an active pharmaceutical ingredient, a drug, or a drug substance is prepared. A predetermined amount of this dispersion is placed into those concavities, and the tablets are dried. In this method, an active ingredient is handled in the form of a dispersion rather than as a solid. In addition, once the tablets are dried, the active ingredient is trapped inside the concavities and remains unexposed to external physical friction. As a result, human contact with powders or dust of active ingredient during the manufacturing and shipping processes may be minimized in a cost-effective manner. The present invention is also directed to the tablets prepared by this method.

### FIELD OF THE INVENTION

The present invention provides a method for manufacturing tablets containing an active ingredient by forming a placebo tablet with a concavity and filling the concavity with an active pharmaceutical ingredient, a drug, or a drug substance. The present invention also provides the tablets thus made.

### BACKGROUND OF THE INVENTION

Conventional methods of manufacturing tablets in the pharmaceutical field may be summarized as follows.

An active pharmaceutical ingredient (“API”), a drug, or a drug substance is mixed with excipients. In a wet-granulation method, this mixture is granulated with water or an organic solvent to form granules and then dried. Alternatively, in a dry-granulation method, the mixture is granulated by using a compaction or slugging method to form granules. The granules thus made are subjected to a milling process to reduce the particle sizes and then mixed with further excipients, if necessary, to form the final mixture or blend. This final blend is compressed to form tablets.

Direct compression is another standard approach for manufacturing tablets. In this method, an API, a drug, or a drug substance is mixed with excipients, and without the granulation process mentioned above, the mixture or blend is compressed into tablets.

However, these conventional methods of manufacturing tablets cause hazardous mixtures of drug powders or dust to be generated within the processing room or facility, posing undesirable health risk to those exposed to this environment.

### BRIEF SUMMARY OF THE INVENTION

The present invention provides a method of manufacturing tablets in which human contact with drug powders or dust during the manufacturing process is minimized, such that the health of the personnel involved may be protected. In other words, the method is beneficial from an environmental safety and health standpoint. Additionally, the present invention is cost-effective, which keeps manufacturing costs low, and therefore is a benefit to consumers.

The method of manufacturing tablets of the present invention includes several steps. First, a placebo blend is prepared. The placebo blend is then compressed into tablets having a concavity or multiple concavities. Some examples of such a concavity are a pinhole and an indentation. Independently of these placebo tablets, a dispersion of an API, a drug, or a drug substance is prepared. The term “dispersion” here means a solution, a suspension, an emulsion, a mixture, or a hot melt. The dispersion may contain multiple APIs, drugs, or drug substances, or multiple dispersions may be prepared with multiple APIs, drugs, or drug substances, for application onto the placebo tablets.

Next, a quantitative amount of the dispersion is applied into the concavities of the tablets. After this dosing step, the tablets are dried, if necessary. Optionally, a binding or coating dispersion may be applied into the concavities in order to help protect or to enhance binding of the API, drug, or drug substance to the tablets.

In this manufacturing process, an API, a drug, or a drug substance is handled in the form of a dispersion, which reduces the amount of an API, a drug, or a drug substance that enters the atmosphere during tablet formulation. Further, once the tablets are dried, the API, drug, or drug substance is trapped inside the concavities. This prevents the API, drug, or drug substance from rubbing off from the tablets during the manufacturing or shipping process. Accordingly, generation of drug powders or dust is suppressed from initial manufacture all the way to end users.

The present invention is also directed to the tablets prepared by this method.

### BRIEF DESCRIPTION OF THE DRAWINGS

FIG. 1 is a cross section of a tablet having one concavity, wherein the concavity is in the form of a pinhole.

FIG. 2 is a cross section of a tablet having two concavities on the opposite faces of the tablet, wherein the concavities are in the form of pinholes.

FIG. 3 is a cross section of a tablet having one concavity, wherein the concavity is in the form of an indentation.

FIG. 4 is a cross section of a tablet having two concavities on the opposite faces of the tablet, wherein the concavities are in the form of indentations.

FIG. 5 is a top view of the face of a tablet having a concavity.

FIG. 6 shows a tablet punch having a projection pin for preparing a tablet having a pinhole.

FIG. 7 shows a tablet punch having a projection cog for preparing a tablet having an indentation.

FIG. 8 shows a schematic of a process in which concave placebo tablets having only one concavity are reoriented so that all the concavities will be facing up in preparation for the dosing step.

FIG. 9 shows a schematic of a process in which an active-ingredient dispersion is applied into the concavities of placebo tablets, and the tablets are dried and then subjected to quality control on a multi-track conveyor.

FIG. 10 shows a schematic of a process in which an active-ingredient dispersion is applied into the concavities of placebo tablets, and the tablets are dried and then subjected to quality control on a single-track conveyor.

### DETAILED DESCRIPTION OF THE INVENTION

In this invention, “tablets” are defined as solid pharmaceutical dosage forms containing one or more active pharmaceutical ingredients (APIs), drugs, or drug substances with suitable excipients and prepared by either compression or molding methods. Examples of tablets formed by compression include sugar-coated tablets, film-coated tablets, enteric-coated tablets, multiple compressed tablets (such as layered tablets and press-coated or dry-coated tablets), controlled-release tablets, solution-purpose tablets, effervescent tablets, compressed suppositories/inserts, buccal tablets, and sublingual tablets. Examples of tablets formed by molding include dispensing tablets and hypodermic tablets.

Examples of a method of preparing a tablet include the wet-granulation method (including high-shear granulation and fluid-bed granulation), the dry-granulation method, and direct compression.

Examples of excipients include substances that help to give satisfactory processing and compression characteristics to the formulation (such as diluents, binders or granulators, lubricants, and glidants) and substances that help to impart additional desirable physical characteristics to the finished tablet (such as disintegrants, coloring agents, and flavoring agents).

Examples of diluents include dicalcium phosphate, calcium sulfate, lactose, cellulose, microcrystalline cellulose, kaolin, mannitol, sodium chloride, dry starch, and powdered sugar.

Examples of binders or granulators include starch, gelatin, sugars (such as sucrose, glucose, dextrose, molasses, and lactose), natural or synthetic gum (such as acacia, sodium alginate, extract of Irish moss, panwar gum, ghatti gum, mucilage of isapol husks, carboxymethylcellulose, methylcellulose, polyvinylpyrrolidone, Veegum, and larch arabogalactan), polyethylene glycol, ethylcellulose, waxes, water, and alcohol.

Examples of lubricants include talc, magnesium stearate, calcium stearate, stearic acid, hydrogenated vegetable oils, and polyethylene glycol (PEG).

Examples of glidants include colloidal silicon dioxide (Cab-o-sil) and talc (asbestos-free).

Examples of disintegrants include starches (such as corn and potato starch and sodium starch glycolate), clays, celluloses (such as croscarmellose), alginates, gums, and cross-linked polymers (such as crospovidone).

Examples of coloring agents include erythrosine, tartrazine, and burnt sugar.

Examples of flavoring agents include peppermint oil, mannitol, and lactose.

In this invention, a “concave” object is defined as an object having the property that there exist two points inside it such that the line segment drawn between them contains points {E} not within the object. A “concavity” of a concave object is defined as a collection of such points {E} that are continuously connected. A concave object may have more than one cavity. An example of a concave object is a round tablet having a pinhole or an indentation, in which case the pinhole or the indentation forms a concavity.

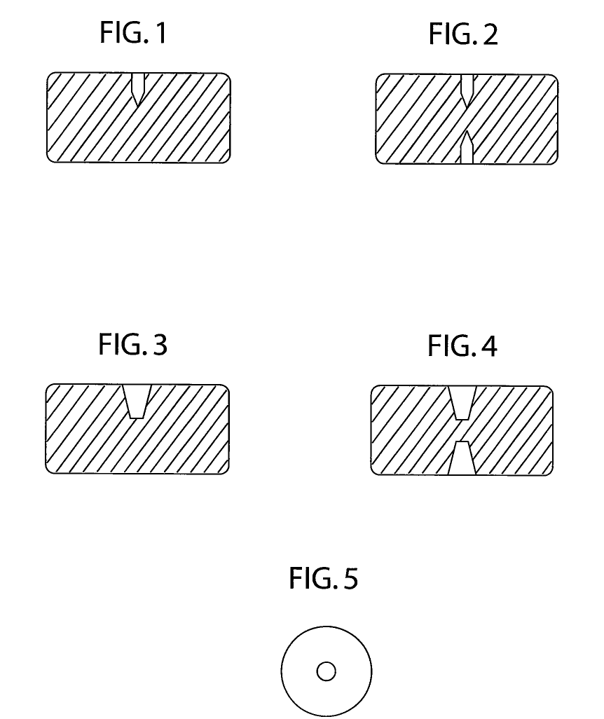

In the method of manufacturing a tablet of the present invention, a placebo blend containing only excipients is first prepared. The placebo blend is then compressed into tablets having a concavity such as a pinhole or an indentation. The tablets may also have two concavities, which may be formed on the opposite faces of a tablet, or they may even have more than two concavities. Examples of such concave tablets are shown in FIGS. 1 through 4.

The shape of concave tablets including their concavities is not particularly limited. For example, it may be discoid, spherical, oval, capsular, oblong, cylindrical, a triangular prism, a square or rectangular prism, a pentagonal prism, a hexagonal prism, an octagonal prism, or any other shape.

The shape of the opening of a concavity is not particularly limited. For example, it may be round, elliptical, triangular, square, diamond-shaped, rectangular pentagonal, hexagonal, octagonal, or any other shape. FIG. 5 shows an example of a round opening.

The depth of a concavity as measured to the bottom is preferably in the range of 0.1 mm or more and 3.0 mm or less, more preferably in the range of 0.5 mm or more and 2.5 mm or less, and even more preferably in the range of 1.0 mm or more and 2.0 mm or less.

The size of the opening of a concavity as measured at its maximum span is preferably in the range of 0.5 mm or more and 5.0 mm or less, more preferably in the range of 1.0 mm or more and 4.0 mm or less, and even more preferably in the range of 1.2 mm or more and 3.0 mm or less.

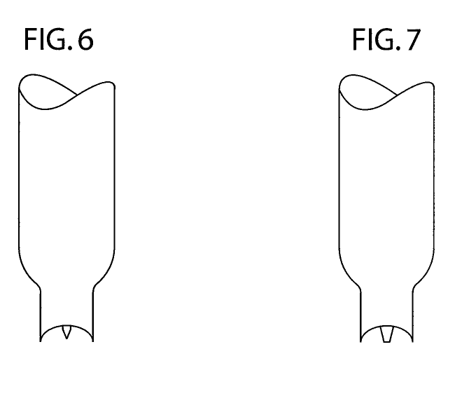

Tablet-compression equipment usually includes a lower punch that fits into a die from the bottom and an upper punch that has a head of the same shape and dimensions and which enters the die volume from the top after the material to be formed into a tablet fills the die volume. Tablets having concavities may be prepared by using a punch with a suitable projection. Examples of such punches for preparing tablets with a pinhole and with an indentation are shown in FIGS. 6 and 7, respectively.

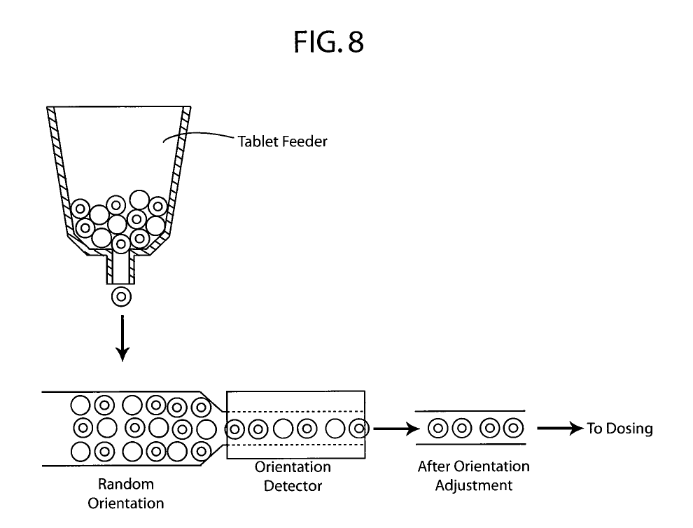

For concave placebo tablets prepared to have only one concavity, the tablets must be reoriented at some point so that all the concavities will be facing up in preparation for the subsequent dosing step. An example of such a process is shown in FIG. 8, in which a single-track conveyor is used for the detection and reorientation steps.

On the other hand, for flat-shaped placebo tablets having concavities on both flat surfaces, the reorientation step is unnecessary. This is because as the tablets are laid down, either one of the two concavities will automatically be facing up. Therefore, forming two concavities on the placebo tablets simplifies the process, even though only one of the two may be used for dosing purpose. This may be accomplished, for example, by having a required projection on both the upper and lower punches of the compression equipment.

Separately from the placebo tablets above, a dispersion of an API, a drug, or a drug substance is prepared. For the purpose of this invention, a “dispersion” is defined as a solution, a suspension, an emulsion, a mixture, or a hot melt. The dispersion may contain multiple APIs, drugs, or drug substances, or multiple dispersions may be prepared with multiple APIs, drugs, or drug substances, for application onto the placebo tablets.

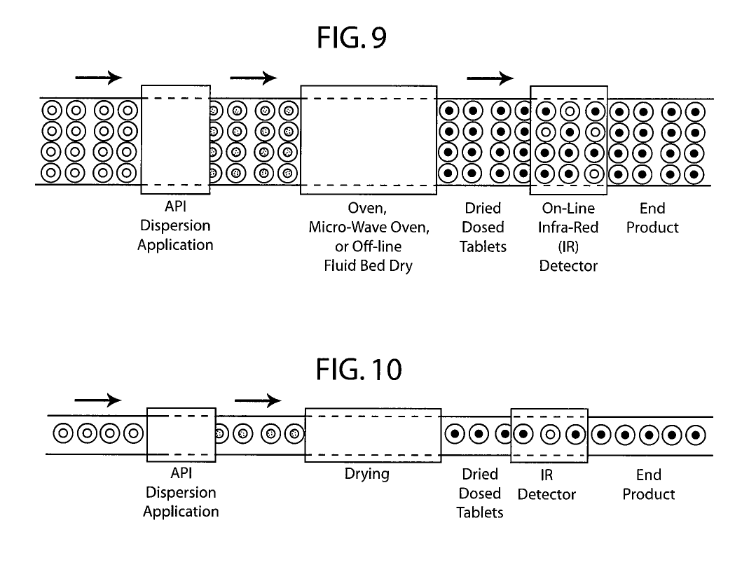

A quantitative amount of the dispersion thus prepared is then applied into the concavities of the placebo tablets. After this dosing step, the tablets are dried if necessary. Examples of drying methods include air dry, oven dry, fluid bed dry, and microwave dry. A schematic of the process is shown in FIGS. 8 and 9 for a multi-track and single-track conveyor, respectively. The figures also contain an on-line infrared detector used for quality control purpose so that undosed or partially dosed tablets may be detected and rejected.

The dosage of the API, drug, or drug substance per tablet is preferably in the range of 0.001 mg or more and 20 mg or less, more preferably in the range of 0.005 mg or more and 10 mg or less, and even more preferably in the range of 0.01 mg or more and 5 mg or less.

Optionally, after the dosing step, a binding or coating dispersion may be applied into the concavities in order to help protect or to enhance the binding of the API, drug, or drug substance to the tablets. The tablets may then be subjected to the drying process.

Film coating of tablets is a common practice in the industry. The present invention allows such practice in a flexible manner. That is, the tablets can be film-coated either before or after an active-ingredient dispersion is applied into the concavities.

The tablets of the present invention may also be designed to have a logo or a code number added for identification purpose.

Finally, the initial tablets may also contain an API, a drug, or a drug substance if desired. In this case, the final tablets will be a combination product containing two or more active ingredients.

Examples of the present invention are provided below. However, the scope of the invention is not limited to these embodiments.

### EXAMPLE 1

### Preparation of Placebo Tablets

The formulation shown below may be used to prepare a placebo tablet.

| Item # | Ingredient | Amount (g) |
|---|---|---|
| 1 | Lactose Monohydrate, NF | 340.0 |
| 2 | Microcrystalline Cellulose, NF | 135.0 |
| 3 | Sodium Starch Glycolate | 20.0 |
| 4 | Colloidal Silicon Dioxide, NF | 2.5 |
| 5 | Magnesium Stearate, NF | 2.5 |

The procedure for preparing a placebo tablet is as follows.
A. Load items #1, 2, 3, and 4 into a blender of a suitable size and mix them for approximately 5 minutes.
B. Pass the mixed powder blend of step A through a #30 mesh screen.
C. Reload the screened mixed powder blend of step B into the blender and mix it for approximately 5 minutes.
D. Pass item #5 through a #30 mesh screen, and add this screened item into the blender of step C and mix them for approximately 5 minutes.
E. Unload this powder blend into a container of a suitable size.
F. Compress the powder blend of step E into tablets using a 5/16″ round plain with a projection pin on an upper punch.
G. Collect the tablets into a container of a suitable size.

### EXAMPLE 2

### Preparation of Placebo Tablets with Film Coating

A film coating can be applied to concave placebo tablets either before or after applying an API, a drug, or a drug substance to the concavity. The formulation shown below may be used to prepare a placebo tablet with a film coating.

| Item # | Ingredient | Amount (g) |
|---|---|---|
| 1 | Placebo Tablets | 980.0 |
| 2 | Opadry White YS-1-7003 | 19.7 |
| 3 | Purified Water, USP | 170.0 |
| 4 | Candelilla Wax Powder, FCC | 0.3 |

The procedure for preparing a placebo tablet with a film coating is as follows.
A. Add item #3 into a container of a suitable size and equipped with a mixer.
B. Slowly add item #2 into the mixing tank of step A, and continue mixing until the coating suspension is uniform.
C. Load tablets of item #1 into a pan coater of a suitable size, and apply the coating suspension of step B onto the tablets.
D. Upon completion of applying the coating suspension, dry the tablets for approximately 10 minutes and then cool down the tablets.
E. Apply item #4 into the pan coater to polish the tablets.
F. Unload the tablets into a container of a suitable size.

### EXAMPLE 3

### Application of API Dispersion to Placebo Tablets

The formulation shown below may be used for applying an API dispersion to placebo tablets.

| Item # | Ingredient | Amount (g) |
|---|---|---|
| 1 | Ethinyl Estradiol, USP | 30.0 |
| 2 | Vitamin E, USP | 30.0 |
| 3 | Povidone, USP (k30) | 0.5 |
| 4 | Alcohol, USP | 190.0 |

The procedure for applying an API dispersion to placebo tablets is as follows.
A. Load item #4 into a container of a suitable size and equipped with a mixer.
B. Add items #1, 2, and 3 into the mixing tank of step A, mix them until they are dissolved, and close and tightly seal the container.
C. Based on the concentration of the API solution, calculate the required amount of solution to be applied in light of the desired dosage strength.
D. Apply the predetermined amount of solution to the placebo tablets through a syringe needle or a proper spraying nozzle tip.
E. Upon drying the tablets (air dry, oven dry, or microwave dry), collect them into a proper container.

### EXAMPLE 4

### Application of API Dispersion to Placebo Tablets

The formulation shown below may be used for applying an API dispersion to placebo tablets.

| Item # | Ingredient | Amount (g) |
|---|---|---|
| 1 | Desogestrel | 150.0 |
| 2 | Ethinyl Estradiol | 30.0 |
| 3 | Vitamin E, USP | 45.0 |
| 4 | Povidone, USP (k30) | 1.0 |
| 5 | Alcohol, USP | 250.0 |

The procedure for applying an API dispersion to placebo tablets is as follows.
A. Load item #5 into a container of a suitable size and equipped with a mixer.
B. Add items #1, 2, 3, and 4 into the mixing tank of step A, mix them until they are dissolved, and close and tightly seal the container.
C. Based on the concentration of the API solution, calculate the required amount of solution to be applied in light of the desired dosage strength.
D. Apply the predetermined amount of solution to the placebo tablets through a syringe needle or a proper spraying nozzle tip.
E. Upon drying the tablets (air dry, oven dry, or microwave dry), collect them into a proper container.

### EXAMPLE 5

### Application of API Dispersion to Placebo Tablets

The formulation shown below may be used for applying an API dispersion to placebo tablets.

| Item # | Ingredient | Amount (g) |
|---|---|---|
| 1 | Levonorgestrel, USP | 100.0 |
| 2 | Vitamin E, USP | 50.0 |
| 3 | Povidone, USP (k30) | 1.0 |
| 4 | Chloroform | 170.0 |
| 5 | Alcohol, USP | 80.0 |

The procedure for applying an API dispersion to placebo tablets is as follows.
A. Load items #4 and 5 into a container of a suitable size and equipped with a mixer.
B. Add items #1, 2, and 3 into the mixing tank of step A, mix them until they are dissolved, and close and tightly seal the container.
C. Based on the concentration of the API solution, calculate the required amount of solution to be applied in light of the desired dosage strength.
D. Apply the predetermined amount of solution to the placebo tablets through a syringe needle or a proper spraying nozzle tip.
E. Upon drying the tablets (air dry, oven dry, or microwave dry), collect them into a proper container.

### EXAMPLE 6

### Application of API Dispersion to Placebo Tablets

The formulation shown below may be used for applying an API dispersion to placebo tablets.

| Item # | Ingredient | Amount (g) |
|---|---|---|
| 1 | Cetirizine Hydrochloride | 800.0 |
| 2 | Povidone, USP (k30) | 8.0 |
| 3 | Purified Water, USP | 700.0 |

The procedure for applying an API dispersion to placebo tablets is as follows.
A. Load item #3 into a container of a suitable size and equipped with a mixer.
B. Add items #1 and 2 into the mixing tank of step A, mix them until they are dissolved, and close and tightly seal the container.
C. Based on the concentration of the API dispersion, calculate the required amount of dispersion to be applied in light of the desired dosage strength.
D. Apply the predetermined amount of dispersion to the placebo tablets through a syringe needle or a proper spraying nozzle tip.
E. Upon drying the tablets (air dry, oven dry, fluid bed dry, or microwave dry), collect them into a proper container.

### EXAMPLE 7

### Application of API Dispersion to Tablets Containing API to Form a Combination Product

The formulations shown below may be used for applying an API dispersion to tablets containing an API to prepare a combination product.

### Part One: Preparation of Tablets Containing API

| Item # | Ingredient | Amount (g) |
|---|---|---|
| 1 | Ibuprofen, USP | 200.0 |
| 2 | Lactose Monohydrate, NF | 120.0 |
| 3 | Avicel PH 102 | 56.0 |
| 4 | Croscamellose Sodium (Ac-Di-Sol) | 20.0 |
| 5 | Colloidal Silicon Dioxide, NF | 2.0 |
| 6 | Magnesium Stearate, NF | 2.0 |

The procedure for preparing the tablets is as follows.
A. Add items #1, 2, 3, 4, and 5 into a mixer of a suitable size and mix them for approximately 10 minutes.
B. Pass the mixture of step A through a screen having a suitable mesh size, add the screened mixture into the same mixer of step A, and mix it for approximately 5 minutes.
C. Add item #6 into the mixer of step B, and mix them for approximately 5 minutes.
D. Unload this powder blend into a container of a suitable size.
E. Compress the powder blend of step D into tablets using desired tablet tooling with a projection pin on an upper punch.
F. Collect the tablets into a container of a suitable size.

### Part Two: Preparation and Application of API Dispersion to Tablets

| Item # | Ingredient | Amount (g) |
|---|---|---|
| 7 | Hydrocodone Bitartrate, USP | 50.0 |
| 8 | Povidone, USP (k30) | 0.5 |
| 9 | Purified Water, USP | 900.0 |

The procedure for applying an API dispersion to the tablets prepared in Part One above is as follows.
G. Load item #9 into a container of a suitable size and equipped with a mixer.
H. Add items #7 and 8 into the mixing tank of step G, mix them until they are dissolved, and close and tightly seal the container.
I. Based on the concentration of the API dispersion, calculate the required amount of dispersion to be applied in light of the desired dosage strength.
J. Apply the predetermined amount of dispersion to the tablets prepared in Part One above through a syringe needle or a proper spraying nozzle tip.
K. Upon drying the tablets (air dry, oven dry, fluid bed dry, or microwave dry), collect them into a proper container.

### EXAMPLE 8

### Application of API Dispersion to Tablets Containing API to Form a Controlled-Release Combination Product

The formulations shown below may be used for applying an API dispersion to tablets containing an API to prepare a controlled-release combination product.

### Part One: Preparation of Controlled-Release Tablets

| Item # | Ingredient | Amount (g) |
|---|---|---|
| 1 | Lactose Monohydrate, NF | 480.0 |
| 2 | Microcrystalline Cellulose, NF | 225.0 |
| 3 | Croscamellose Sodium (Ac-Di-Sol) | 32.0 |
| 4 | Magnesium Stearate, NF | 4.0 |
| 5 | Pseudoephedrine HCl, USP | 300.0 |
| 6 | Hydroxypropyl Methylcellulose 2208, USP | 420.0 |
| | (Methocel K100 M Premium) | |
| 7 | Lactose Monohydrate, NF | 60.0 |
| | (Modified Spray Dried) | |
| 8 | Colloidal Silicon Dioxide, NF (Cab-O-Sil) | 4.0 |
| 9 | Magnesium Stearate, NF | 6.0 |

The procedure for preparing the controlled-release tablets is as follows.
A. Pass items #1, 2, and 3 through a screen having a suitable mesh size, add the screened items into a mixer of a suitable size, and mix them for approximately 5 minutes.
B. Add item #4 into the mixer of step A, and mix them for approximately 5 minutes.
C. Unload this placebo powder blend into a container of a suitable size.
D. Pass items #5, 6, 7, and 8 through a screen having a suitable mesh size, add the screened items into a mixer of a suitable size, and mix them for approximately 5 minutes.
E. Add item #9 into the mixer of step D, and mix them for approximately 5 minutes.
F. Unload this controlled-release powder blend into a container of a suitable size.
G. Set up a tablet press machine equipped with double-layer function, wherein a lower punch is plain tooling and an upper punch is with a projection pin.
H. Compress double-layer tablets using the placebo powder blend from step C and the controlled-release powder blend from step F, wherein the tablets have a pinhole located on the placebo side.
I. Collect the tablets into a container of a suitable size.

### Part Two: Preparation and Application of API Dispersion to Controlled-Release Tablets

| Item # | Ingredient | Amount (g) |
|---|---|---|
| 10 | Cetirizine Hydrochloride | 400.0 |
| 11 | Povidone, USP (k30) | 4.0 |
| 12 | Purified Water, USP | 350.0 |

The procedure for applying an API dispersion to the controlled-release tablets prepared in Part One above is as follows.
J. Load item #12 into a container of a suitable size and equipped with a mixer.
K. Add items #10 and 11 into the mixing tank of step J, mix them until they are dissolved, and close and tightly seal the container.
L. Based on the concentration of the API dispersion, calculate the required amount of dispersion to be applied in light of the desired dosage strength.
M. Apply the predetermined amount of dispersion to the controlled-release tablets prepared in Part One above through a syringe needle or a proper spraying nozzle tip.
N. Upon drying the tablets (air dry, oven dry, fluid bed dry, or microwave dry), collect them into a proper container.

What is claimed is:

1. A tablet comprising:
a component selected from the group consisting of an active pharmaceutical ingredient, a drug, and a drug substance; and
an excipient;
wherein:
the tablet has a concavity or concavities; and
the component is disposed in its entirety within the concavity or at least one of the concavities of the tablet.

2. The tablet according to claim 1, wherein the tablet has more than one concavities.

3. The tablet according to claim 2, wherein the tablet has only two concavities and the two concavities are on the opposite sides of the tablet.

4. The tablet according to claim 3, wherein the component is disposed in its entirety within only one of the two concavities.

5. The tablet according to claim 1, wherein the concavity within which the component is disposed in its entirety has a depth as measured to the bottom in the range of 0.1 mm or more and 3.0 mm or less.

6. The tablet according to claim 5, wherein the depth of the concavity is in the range of 0.5 mm or more and 2.5 mm or less.

7. The tablet according to claim 6, wherein the depth of the concavity is in the range of 1.0 mm or more and 2.0 mm or less.

8. The tablet according to claim 1, wherein the opening of the concavity within which the component is disposed in its entirety has a size as measured at its maximum span in the range of 0.5 mm or more and 5.0 mm or less.

9. The tablet according to claim 8, wherein the size of the opening of the concavity is in the range of 1.0 mm or more and 4.0 mm or less.

10. The tablet according to claim 9, wherein the size of the opening of the concavity is in the range of 1.2 mm or more and 3.0 mm or less.

11. The tablet according to claim 1, wherein the amount of the component disposed in its entirety within the concavity or at least one of the concavities of the tablet is in the range of 0.001 mg or more and 20 mg or less.

12. The tablet according to claim 11, wherein the amount of the component is in the range of 0.005 mg or more and 10 mg or less.

13. The tablet according to claim 12, wherein the amount of the component is in the range of 0.01 mg or more and 5 mg or less.

14. A method of manufacturing a tablet, comprising the steps of:
preparing a tablet having a concavity or concavities; and
disposing, into the concavity or at least one of the concavities of the tablet, a dispersion comprising a component selected from the group consisting of an active pharmaceutical ingredient, a drug, and a drug substance.

15. The method according to claim 14, wherein the tablet formed in the preparation step has more than one concavities.

16. The method according to claim 15, wherein the tablet formed in the preparation step has only two concavities and the two concavities are on the opposite sides of the tablet.

17. The method according to claim 14, wherein in the disposing step, the amount of the component contained in the dispersion disposed into the concavity or at least one of the concavities of the tablet is in the range of 0.001 mg or more and 20 mg or less.

18. The method according to claim 14, wherein in the disposing step, the amount of the component contained in the dispersion disposed into the concavity or at least one of the concavities of the tablet is in the range of 0.005 mg or more and 10 mg or less.

19. The method according to claim 14, wherein in the disposing step, the amount of the component contained in the dispersion disposed into the concavity or at least one of the concavities of the tablet is in the range of 0.01 mg or more and 5 mg or less.
